(19) United States
(12) Patent Application Publication
(10) Pub. No.: US 2025/0017535 A1
(43) Pub. Date: Jan. 16, 2025

(54) SYSTEMS AND METHODS FOR ASSESSING A PATIENT AND PREDICTING PATIENT OUTCOMES

(71) Applicant: Gaitly Inc., Calgary (CA)

(72) Inventors: Thomas TRUONG, Calgary (CA); John BUGO, Canmore (CA); Salim SAMANANI, Calgary (CA); Wei TAM, Calgary (CA); Tanya JOON, Edmonton (CA)

(21) Appl. No.: 18/771,554

(22) Filed: Jul. 12, 2024

Related U.S. Application Data
(60) Provisional application No. 63/513,418, filed on Jul. 13, 2023.

(51) Int. Cl. A61B 5/00; A61B 5/11; G16H 10/60; G16H 50/30

(52) U.S. Cl. CPC A61B 5/7267; A61B 5/1121; A61B 5/1128; A61B 5/4088; A61B 5/7275; G16H 10/60; G16H 50/30

(57) ABSTRACT

Systems and methods for assessing a patient and predicting patient outcomes are provided. The system may comprise an electronic medical records (EMR) subsystem and a data analysis and prediction subsystem. The method may comprise: receiving video and audio data of a patient assessment including at least one gait or balance assessment activity and/or at least one cognitive assessment activity; processing the video data to obtain pose information; processing the audio data to obtain speech information; extracting at least one gait or balance measurement from the pose information; extracting at least one cognitive measurement or cognitive normative score from the speech information; and generating, via a machine learning model, a prediction of a patient outcome using the at least one gait or balance measurement and/or the at least one cognitive measurement or cognitive normative score as input.

### CROSS-REFERENCE TO RELATED APPLICATION

The present disclosure claims priority to U.S. Provisional No. 63/513,418, filed Jul. 13, 2023, the entire content of which is herein incorporated by reference.

### TECHNICAL FIELD

The present disclosure relates to medical assessments. More particularly, the present disclosure relates to systems and methods for assessing a patient and predicting patient outcomes relating to gait, balance, and cognition.

### BACKGROUND

A variety of standard assessments for patient gait, and balance have been developed, including the Dynamic Gait Index (DGI), the “Timed Up and Go” (TUG) test, the Balance Error Scoring System (BESS), and the Functional Gait Assessment (FGA). These assessments involve the patient performing a specific task or activity while a clinician observes them. The clinician may then assign a normative score for that activity. For example, the FGA consists of 10 activities including the FGA “gait level surface” in which the patient walks at a normal speed on a level surface and the clinician assigns a score or between 0 and 3, with 0 indicating severe impairment and 3 indicating normal movement. The scores from each of the FGA activities are then added to come to a final score. The final score may help the clinician assess the patient's risk of falling and can be used to diagnose and monitor a variety of conditions including neurodegenerative diseases, brain injuries, and more.

However, many of the current clinical tests that can be performed at home or in a typical clinical setting are not sensitive or precise enough to predict risk, or monitor disease or treatment progression, for example, for falls, neurodegenerative conditions like Alzheimer's dementia, or traumatic brain injuries (also known as concussions). Gait and balance assessments currently used in clinical practice rely on clinical observation alone, supplemented by clinical skill and judgement. They cannot achieve a level of measurement precision that goes beyond what a human can reasonably observe or measure in a clinical setting. Many of the scoring criteria are also subjective with intra-and inter-rater reliability issues. As a result of the lack of reliability and precision, assessments cannot be carried out by patients themselves (i.e., they rely on the presence of a trained clinician). Other limitations of current methodologies include clinical risk stratification being quite crude or broad (e.g., high, medium, low), subtle changes in neuromotor health go undetected, and false positive and false negative outcomes are common.

The sensitivity, precision, reliability, and objectivity of gait and balance measurements and analyses can be increased with use of specialized equipment such as wearables, three-dimensional (3D) motion capture sensors, pressurized sensor walkways, or depth cameras, to capture data as the patient moves. However, such equipment is expensive and isn't accessible for typical clinical settings or for the patient to be able to perform the tests themselves, in their home. These types of equipment also have their own limitations, for example, pressure sensor walkways cannot measure postural angles or limb movements. In addition, current technologies are limited to only one type of assessment (e.g. gait/balance assessments) and are not used to assess other factors that contribute to overall patient outcomes.

In addition, the data captured during patient assessments is complex and may require considerable processing power to generate usable information and predictions. Current systems may also be limited to more quantitative measurements and may not be able to perform more qualitative assessments that contribute to more complex patient outcomes such as fall risk and disease progression.

### SUMMARY

In one aspect, there is provided a computer-implemented method comprising: receiving, from a user device, video data and audio data of a patient assessment, wherein the patient assessment comprises at least one gait or balance assessment activity and/or at least one cognitive assessment activity performed by a patient; processing the video data to obtain pose information, wherein the pose information comprises a plurality of joint points representing the patient; processing the audio data to obtain speech information in the form of transcribed text, wherein the speech information comprises semantic information and/or speech features; extracting at least one gait or balance measurement from the pose information; extracting at least one cognitive measurement or cognitive normative score from the speech information; and generating, via a machine learning model, a prediction of a patient outcome using the at least one gait or balance measurement and/or the at least one cognitive measurement or cognitive normative score as input, wherein the machine learning model is trained using training data comprising patient outcomes in association with a plurality of previous gait or balance assessments and cognitive assessments.

In some embodiments, the method further comprises determining a gait or balance normative score for the at least one gait or balance assessment activity based on the at least one gait or balance measurement; and wherein the input for the machine learning model further comprises the gait or balance normative score.

In some embodiments, the cognitive normative score is extracted from the semantic information of the speech information.

In some embodiments, the method further comprises determining at least one additional cognitive normative score based on the at least one cognitive measurement; and wherein the input for the machine learning model further comprises the at least one additional cognitive normative score.

In some embodiments, the input for the machine learning model further comprises at least one of the pose information and the speech information.

In some embodiments, the patient outcomes in the training data comprise patient-reported outcomes.

In some embodiments, the at least one gait or balance measurement comprises at least one of: step and stride measurements; gait cycle phases for each leg;

estimation of trunk sway; asymmetry detection during movement; joint angles; and joint velocities.

In some embodiments, at least one of processing the video data and extracting the at least one gait or balance measurement are performed using at least one machine learning model trained using sensor data collected from physical sensors during a plurality of gait or balance assessments.

In some embodiments, at least one machine learning model is a large-language model.

In some embodiments, processing the audio data further comprises identifying one or more speech cues in the speech information and assigning a respective timestamp to each of the one or more speech cues.

In some embodiments, processing the video data further comprises cropping or segmenting the video data based on the respective timestamp of at least one speech cue of the one or more speech cues.

In some embodiments, the at least one gait or balance measurement comprises a timed measurement and one speech cue of the one or more speech cues indicates the start of the at least one gait or balance assessment activity, and wherein processing the video data further comprises assigning an activity start timepoint to the video data based on the respective timestamp of the one speech cue.

In some embodiments, the timed measurement comprises a measured time lag between the activity start timepoint and a movement start timepoint, wherein the movement start timepoint is assigned based on the pose information.

In some embodiments, the method further comprises determining a respective cost factor for each of the at least one gait or balance assessment activity and the at least one cognitive assessment activity in comparison to another gait or balance assessment activity and cognitive assessment activity, respectively, performed independently.

In another aspect, there is provided a computer-implemented method comprising: receiving, from a user device, video data and audio data of a patient assessment, wherein the patient assessment comprises at least one gait or balance assessment activity; processing the audio data to obtain speech information in the form of transcribed text, wherein processing the audio data further comprises identifying one or more speech cues in the speech information and assigning a respective timestamp to each of the one or more speech cues; assigning one or more timepoints to the video data based on the respective timestamps of the one or more speech cues to define at least one segment; processing the video data of the at least one segment to obtain pose information, wherein the pose information comprises a plurality of joint points representing the patient; extracting at least one gait or balance measurement from the pose information; and generating, via a machine learning model, a prediction of a patient outcome using the at least one gait or balance measurement as input, wherein the machine learning model has been trained using training data comprising patient outcomes in association with a plurality of previous gait or balance assessments.

In some embodiments, extracting at least one cognitive measurement or cognitive normative score from the speech information, and wherein the input for the machine learning model further comprises the at least one cognitive measurement or cognitive normative score.

In some embodiments, the at least one gait or balance assessment activity is performed simultaneously with at least one cognitive assessment activity; the method further comprises determining a cost factor for the at least one gait or balance assessment activity in comparison to another gait or balance assessment activity performed independently; and the input for the machine learning model further comprises the cost factor.

In another aspect, there is provided a system comprising: an electronic medical records (EMR) subsystem comprising one or more databases to receive and store video and audio data of patient assessments and patient assessment results; and a data analysis and prediction subsystem comprising one or more processors executing processor-readable instructions causing the one or more processors to perform any embodiments of the methods disclosed herein.

Other aspects and features of the present disclosure will become apparent, to those ordinarily skilled in the art, upon review of the following description of specific embodiments of the disclosure.

### BRIEF DESCRIPTION OF THE DRAWINGS

Some aspects of the disclosure will now be described in greater detail with

reference to the accompanying drawings. In the drawings:

FIG. 1 is a schematic of an example system for assessing a patient, according to some embodiments;

FIG. 2 is a block diagram of a user device of the system of FIG. 1;

FIG. 3 is a schematic of an electronic medical records (EMR) subsystem of the system of FIG. 1;

FIG. 4A is a block diagram of a data analysis and prediction subsystem of the system of FIG. 1;

FIG. 4B is a block diagram of the machines learning modules of the subsystem of FIG. 4A;

FIG. 5 is a schematic of an example training structure for the machine learning modules of FIG. 4B;

FIG. 6 is a flowchart of an example method for assessing a patient, according to some embodiments; and

FIG. 7 is a flowchart of an example method for training machine learning models, according to some embodiments.

### DETAILED DESCRIPTION

Generally, the present disclosure provides systems and methods for assessing a patient and predicting patient outcomes. The methods and systems may employ one or more machine learning models.

As used herein the terms “a”, “an,” and “the” may include plural referents unless the context clearly dictates otherwise.

As used herein, “gait or balance assessment” refers to any test of a patient's ability to walk and/or maintain their position and is inclusive of assessments for gait, balance, posture, stability, mobility, and more. The term “gait or balance” is used herein interchangeably with “gait/balance” and it will be understood that some gait or balance assessments may assess both gait and balance. A “standard gait or balance assessment” refers to a gait or balance assessment with standardized activities and scoring including, but not limited to, the Dynamic Gait Index (DGI), the “Timed Up and Go” (TUG) test, the Balance Error Scoring System (BESS), and the Functional Gait Assessment (FGA). A “gait or balance assessment activity” or “gait or balance task” refers to any discrete activity performed by a patient as part of a gait or balance assessment. For example, the FGA includes ten discrete activities including gait level surface, change in gait speed, gait with horizontal head turns, etc.

As used herein, “cognitive assessment” refers to any test of a patient's cognitive ability including, but not limited to, mental status, concentration, speech, and memory. A “standard cognitive assessment” refers to a cognitive assessment with standardized activities and scoring including, but not limited to, Serial Subtractions, the Montreal Cognitive Assessment (MoCA), and others. A “cognitive assessment activity” or “cognitive assessment task” refers to any discrete activity performed by a patient as part of a cognitive assessment. Other examples of cognitive assessment activities include, but are not limited to: asking the patient to name as many words as they can starting with a particular letter of the alphabet: reciting a list of objects provided to them by a care provider after a certain time; recalling a list of numbers backwards, etc. Although speech ability is included in the term “cognitive assessment” herein, it will be understood that speech deficits may have both cognitive and physical components.

As used herein, a “dual-task assessment” or “dual-tasking” refers to any test that combines at least one gait or balance assessment activity and at least one cognitive assessment activity at the same time. As one example, a TUG test can be performed simultaneously with Serial Subtractions. The patient's performance in a dual-task assessment may be indicative of more subtle changes or abnormalities than could be identified through either activity alone. For example, a patient may perform slightly worse in a TUG test as part of a dual-task assessment than a TUG test performed independently. This decrease in performance is generally referred to as the “cost” of dual-tasking and may be the result of the dual-task exceeding the cognitive resources available to the patient, which may depend on age, health condition, etc. Dual-tasking has shown promise in recent years to be a more sensitive predictor of neuromotor deficits and adverse patient outcomes.

Collectively, reference to a “patient assessment” herein is intended to be inclusive of gait, balance, cognitive, and dual-task assessments and a “patient assessment activity” is any gait, balance, cognitive, and dual-task activity.

As used herein, “patient” refers to any individual being assessed by an embodiment of the systems and methods disclosed herein. A “care provider” may be used interchangeably to refer to an individual providing care to the patient, including but not limited to, a physician, a nurse, a physiotherapist, an occupational therapist, any other health professional, or a family member or other individual associated with the patient.

As used herein, a “user” refers to any individual that is utilizing an embodiment of the system disclosed herein. The user may be a patient, a care provider, or any other individual authorized to use the system as discussed below.

As used herein, “machine learning model” refers to any model generated by machine learning algorithms based on training data to make determinations without being explicitly programmed to do so. Machine learning models may include, but are not limited to, artificial neural networks, support vector machines, decision trees, regression analysis, Bayesian networks, and genetic algorithms. As used herein, “deep learning” or “deep learning model” refers to a subset of machine learning models that use artificial neural networks. A “large-language model” refers to a type of deep learning model that utilizes large language datasets to recognize and interpret human language text. A “multimodal” large-language model refers to a large-language model which is capable of recognizing and interpreting multiple data inputs like text, video, image, audio and tables.

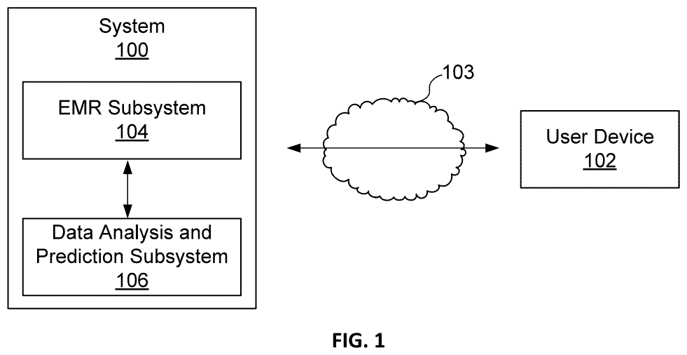

FIG. 1 is a schematic of an example system 100 for assessing a patient, according to some embodiments.

The system 100 in this embodiment comprises two subsystems: an electronic medical record (EMR) subsystem 104 and a data analysis and prediction (DAP) subsystem 106. Each subsystem 104 and 106 may be in the form of one or more servers. As used herein “server” refers to any network equipment comprising circuitry, hardware, and/or software for performing the functions described herein.

The system 100 is operable to communicate with a user device 102 over one or more communication networks 103 such as the Internet. As used herein, “user device” refers to any device capable of communication over a communication network including, but not limited to, personal computers, laptops, and mobile communication devices such as mobile phones, smart phones, tablets, and smart watches. In some embodiments, the “user device” may be a combination of devices, such as a personal computer and a camera, such as a webcam.

The communication network 103 may comprise a wired or a wireless network. The wireless network may comprise a mobile device communication network, a satellite communication network, a local area network (LAN) such as Wi-Fi and/or any other suitable wireless network.

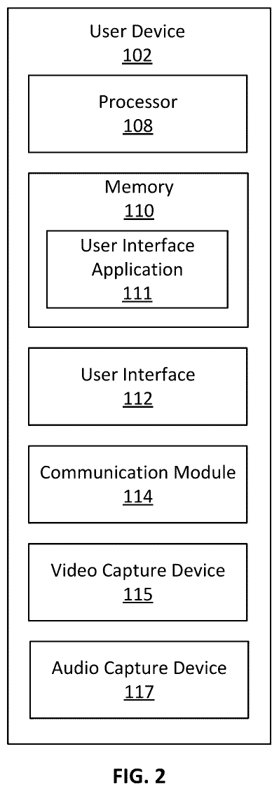

The user device 102 will be described in more detail with reference to FIG. 2. The user device 102 in this embodiment comprises a processor 108, a memory 110, a user interface 112, a communication module 114, at least one video capture device 115, and at least one audio capture device 117.

The memory 110 is operatively connected to the processor 108. The memory 110 may store processor-executable instructions therein that, when executed, cause the processor 108 to implement one or more of the functions described herein.

A user interface application 111 may be stored in the memory 110 and implemented via the user interface 112. The user interface application 111 may be provided as a web browser application, a mobile application, a desktop application, and/or any other suitable type of application. The user interface application 111 may be configured to prompt the user to perform and record a patient assessment activity and upload video and audio data of the completed assessment activity.

In some embodiments, the user interface application 111 may provide feedback to the user during the patient assessment activity via the output components of the user interface 112 discussed below. In some embodiments, the feedback may comprise visual and/or audio cues. For example, the feedback may be an audible alert that is generated when the patient moves out of frame of the video capture device 115. In some embodiments, the visual or audio cue may include specific instructions for the patient such as a voice that explains the next action for the patient to take. The feedback may be provided in real-time such that the user does not need to stop the recording to take the action indicated by the feedback.

The user interface application 111 may also allow users to access assessment results stored in the EMR subsystem 104 and perform other actions depending on the user's permission level, as discussed in more detail below. As used herein, “assessment results” is inclusive of measurements and normative scores assigned to patient assessment activities as well as patient outcome predictions, and any other analyses performed using patient data.

The user interface 112 is configured to display information to the user and receive user input. The user interface may comprise at least one input component and at least one output component. The input component may comprise, for example, at least one of a touchscreen, a keyboard, a keypad, a mouse, a trackball, a stylus, a navigation pad, a voice input device, or any other suitable type of input device. The output component may comprise, for example, at least one of a display screen, a projector, a voice output device, or any others suitable type of output device. In some embodiments, the user interface 112 comprises a combined display and input component, such as a touchscreen.

The communication module 114 may be configured for both short-range communication and long-range communication. For short-range communication, the communication module 114 may comprise, for example, a Bluetooth transceiver. In some embodiments, the transceiver comprises both a transmitter and a receiver sharing common circuitry. In other embodiments, the transceiver comprises a separate transmitter and receiver. For long range communications, the communications module 114 may comprise a transceiver configured to send and receive communications over a communication network such as the Internet.

The video capture device 115 may be configured to capture video data of a patient performing at least one patient assessment activity. As used herein, “video data” is inclusive of data comprising one or more images and/or image-based sequences. The video capture device 115 may comprise one or more RGB sensors such that the RGB video data is recorded. The video capture device 115 may be an integrated camera in the user device 102, such as the integrated camera of a smart phone or tablet. Alternatively, the video capture device 115 may be a separate camera or another device operable to communicate with the user device 102 via the communication module 114. For example, the video capture device 115 may comprise one or more webcams in communication with a personal computer. The video capture device 115 may be a single camera, which may provide all the video data for the normative score determination and patient outcome predictions discussed below. However, embodiments with multi-camera set ups are also contemplated, such as for more research-focused practitioners seeking more detailed results.

The audio capture device 117 may be configured to capture audio data of a patient performing at least one assessment activity. As used herein, “audio data” is inclusive of audio signals that have been encoded in digital form in any audio coding format and may be uncompressed or compressed. The audio capture device 117 may comprise, for example, a microphone. The audio capture device 117 may be integral with the user device 102 or may be a separate device operable to communicate with the user device 102 via the communication module 114. The audio capture device 117 may be integrated with the video capture device 115 or may be a separate device. In some embodiments, the audio capture device 117 is a single microphone that captures all of the audio data for the normative score determination and patient outcome predictions discussed below. However, embodiment with multiple microphone set ups are also contemplated, such as in combination with the multi-camera set ups mentioned above.

The processor 108 may be operatively connected to the video capture device 115 and audio capture device 117 and may be configured to receive and process video and audio data therefrom. The video and audio data may be transmitted to the EMR subsystem 104 of the system 100 via the communication network 103 using the communication module 114.

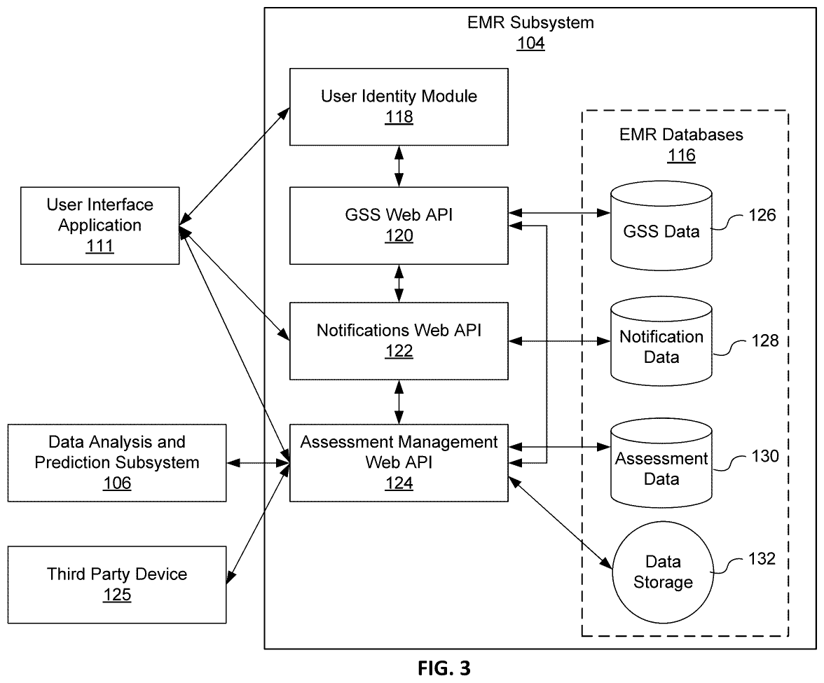

The EMR subsystem 104 will be discussed in more detail with reference to FIG. 3. The EMR subsystem 104 is configured to receive and store the video and audio data from the user device 102 as well as the assessment results generated by the data and analysis subsystem 106. The EMR subsystem 104 is also configured to provide access control and managed permissions for users of the system 100.

The EMR subsystem 104 in this embodiment comprises a user identity module 118, a general security and settings (GSS) web API 120, a notifications web API 122, and an assessment management web API 124. As used herein, “API” refers to an application program interface that provides programmatic access to a software system, and a “web API” is an API for applications hosted on the internet.

The EMR subsystem 104 may further comprise one or more EMR databases 116. In this embodiment, the EMR subsystem 104 comprises a GSS database 126, a notification database 128, and an assessment database 130. The EMR databases 116 may utilize data storage 132.

In some embodiments, the EMR subsystem 104 is hosted in the cloud using a cloud computing platform. In some embodiments, the cloud computing platform is Microsoft's Azure platform. In other embodiments, any other suitable cloud computing platform may be used. The modules and APIs of the EMR subsystem 104 may be implemented by software running on cloud processors and the EMR databases 116 may involve cloud-provided storage. In some embodiments, the EMR databases 116 are implemented using a relational database management system, such as Microsoft SQL Server. In some embodiments, the EMR subsystem 104 is configured to use health record interoperability and messaging standards such as, for example, HL7 and FIHR, which allows communication with other health information systems.

The user identity module 118 is operatively connected to the user interface application 111 and the GSS web API 120. The user identity module 118 is configured to manage user identities and allow users to access their respective user accounts. The user identity module 118 is in communication with the user device 102 and may prompt the user, via the user interface application 111, to enter their login credentials. A user's login credentials may include a user name and a password. The user may be prompted to select a suitably secure password, such as one with a combination of letters, numbers, and special characters. In some embodiments, the user identity module 118 authenticates the user, for example, by two-factor authentication. In some embodiments, the user identify module 118 uses a cloud-based authentication service, such as Microsoft Azure Active Directory B2C.

The GSS web API 120 is operatively connected to the user identify module 118, the notifications web API 122 and the assessment management web API 124. The GSS web API 120 is configured to manage account permissions at both the individual user and organizational levels. The GSS web API 120 may thereby allow users to manage permissions related to their own account or multiple accounts within an organization. The GSS web API 120 may assign one or more designations of account permissions to each user. In some embodiments, there are three primary designations of account permissions: patient, care provider, and organization administrator. A given user account can have multiple designations.

The designation assigned to a user account determines the permissions that the user has with respect to accessing and modifying data in the EMR subsystem 104 via the user interface application 111. For example, a user with patient permissions may have the ability to: access a patient record containing the user's own assessment results, record and upload new assessments; create summary documents of the user's own assessments; and/or add care providers as designated users to receive the user's assessment results. Care provider permissions may give the user the ability to: manage patient records; view assessment results for patients under their care; record and upload new assessments for patients under their care; create clinical documentation and insurance claims for specific patients; and/or send push notifications to the user device 102 of a patient to prompt the patient to perform a self-assessment or to come into the care provider's clinic.

Organization administrator permissions may give the user the ability to manage permission for users within the organization, including patients and care providers. Users with organization administrator permissions may also be able to manage their access to the system 100, for example, by managing a subscription.

The GSS web API 120 may also be configured to generate a log when any user is authenticated via the user identity module 118 and the GSS web API 120. A log may also be generated when any changes are made to a user account or the organization's subscription. Such logs may be stored in the data storage 132 and may be used for auditing purposes regarding user access to the system 100.

The GSS web API 120 is operatively connected to the GSS database 126. The GSS database stores information about users, organizations, and their permissions and settings. For a given user, this information may include: the organization to which the user belongs; settings that the user has configured for their account; security features enabled for the user's organization; permission levels for within the user's organization; permission levels related to the organization's subscription; and auditing logs about user access and any changes that occurred during the access.

The notifications web API 122 is configured to provide asynchronous communications between the user interface application 111 and the assessment management web API 124. The notifications web API 122 is also operatively connected to the GSS web API 120. The notifications web API 122 may be implemented using SignalR or any other suitable API software.

The notifications web API 122 may be configured to push notifications to the user interface application 111 of a given user device 102 automatically in response to an assessment-related event. The assessment-related event may include a new assessment being uploaded, a new analysis or prediction being completed by the data analysis and prediction subsystem 106, or any other suitable assessment-related event.

The notification may inform the user of the assessment-related event and, in some embodiments, may indicate an action to be taken by the user. For example, a new notification may be pushed to a patient's care provider in response to the patient uploading a new self-assessment. The notification may indicate that the care provider should review the assessment results. In some embodiments, the notification may also indicate if patient intervention is recommended, for example, if the assessment results show that the patient's condition is worsening. Alternatively, or additionally, a notification may also be sent to the patient to prompt the patient to contact their care provider. Notifications may also be sent to patients indicating when it is time to perform a regularly scheduled self-assessment.

The notifications web API 122 may also be configured to push notifications to a user in response to input from a different user. For example, a patient may submit a request, via their user interface application 111, and a corresponding notification may be sent to the care provider's user interface application 111 (or vice versa). Such requests may include a request to review recent assessment results, schedule an appointment, etc.

The notifications web API 122 may also generate a log when any requests for access to a patient's assessment results are received. In some embodiments, the notifications web API 122 may also generate a notification in response to such requests, which may be sent to the patient's care provider, an organizational administrator, or another party such as a regulatory body.

The notifications web API 122 is operatively connected to the notification database 128. The notification database 128 may contain information about push notifications sent between different modules, including logs of when requests to access patient assessment results are received. The notification database 128 may also contain information regarding the occurrence of assessment-related events that resulted in a notification being sent.

The assessment management web API 124 is operatively connected to the user interface application 111, the GSS web API 120, the notifications web API 122, and the data analysis and prediction subsystem 106. The assessment management web API 124 is also operatively connected to the assessment database 130 and data storage 132. The assessment management web API 124 is configured to handle functionality relating to managing video and audio data and assessment results.

The assessment management web API 124 is configured to receive video and audio data recorded by the user device 102, which is then stored in the assessment database 130 and data storage 132. The assessment management web API 124 may also transmit the video and audio data to the data analysis and prediction subsystem 106, where the data is analyzed to generate assessment results, as discussed in more detail below. The assessment results are then received by the assessment management web API 124 and are also stored in the assessment database 130.

The assessment management web API 124 is also configured to allow users to view and manage information regarding patient assessments via the user interface application 111. The user permissions established by the GSS web API determine the activities that the user can perform.

For a patient, the assessment management web API 124 may allow the user to perform various activities including, but not limited to: record and upload patient assessment activities for analysis; receive assessment results; retrieve and view their historical assessment results and trends over time; and/or view detailed dashboards of their assessment results. For a care provider, the assessment management web API 124 may allow the user to perform various activities including, but not limited to: sort and filter patient records; view detailed dashboards containing assessment results and analyses; create pre-treatment gait, balance, or cognitive baselines and track pose-treatment recovery; and send assessment results to other EMR systems using interoperability and messaging standards like HL7 or FIHR.

In some embodiments, the assessment management web API 124 is also operatively connectable to a third-party device 125. The third-party device may be a smart phone, personal computer, etc. that does not have the user interface application 111. In some embodiments, the assessment management web API 124 is configured to receive video and audio data from the third-party device 125 and transmit the video and audio data to the data analysis and prediction subsystem 106 for analysis. The assessment management web API 124 may also allow the user of the third-party device 125 to view and manage assessment results generated from the video and audio data and/or any other relevant information regarding a particular patient. For example, a care provider such as a physiotherapist may have video and audio data of a patient performing a patient assessment activity on their own device that was not acquired through the user interface application 111. The care provider can send the video and audio data to the system 100 and view the results via the assessment management web API 124.

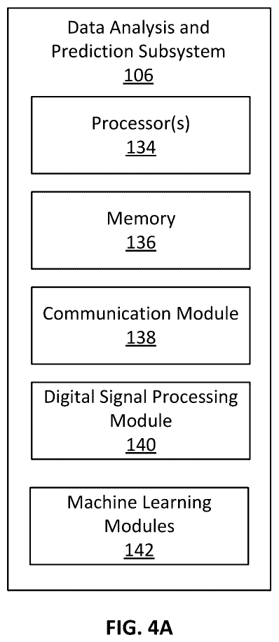

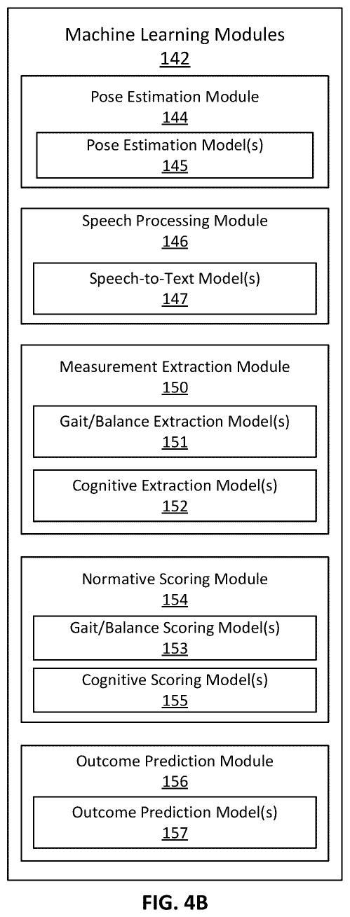

The data analysis and prediction subsystem (DAP) 106 will be discussed with reference to FIGS. 4A and 4B.

As shown in FIG. 4A, the DAP subsystem 106 comprises at least one processor 134, a memory 136, a communication module 138, a digital signal processing (DSP) module 140, and machine learning modules 142. FIG. 4B provides additional details on the machine learning modules 142. In this embodiment, the machine learning modules 142 include: a pose estimation module 144, a speech processing module 146, a measurement extraction module 150, a normative scoring module 154, and an outcome prediction module 156.

The memory 136 is operatively connected to the processor(s) 134. The memory 136 stores processor-executable instructions therein that, when executed, cause the processor(s) 134 to implement one or more of the functions and/or methods described herein. The memory 136 may be internal or external to the processor 134.

The communication module 138 may comprise one or more transceivers. configured to send and receive communications over one or more communication networks such as any of the communication networks discussed above. In some embodiments, the transceiver comprises both a transmitter and a receiver sharing common circuitry. In other embodiments, the transceiver comprises a separate transmitter and receiver. The communication module 138 allows the DAP subsystem 106 to receive data and instructions from the EMR subsystem 104 and transmit information and instructions thereto.

The DSP module 140 and the machine learning modules 142 may each be implemented as a processor (such as the processor 134) configured to perform the functions described below. Each module may be implemented as a memory (such as the memory 136) containing instructions for execution by a processor (such as the processor 1134), by hardware, or by a combination of instructions stored in a memory and additional hardware, to name a few examples.

The DSP module 140 is configured to employ digital signal processing methodologies to reduce noise from the output of one or more of the machine learning modules 142. The DSP module 140 may employ combinations of digital signal processing methodologies including Savitsky-Golay, Kaiser-Bessel, and Weiner filters to minimize the effect of noise on outputs from the pose estimation module 144 and/or the speech processing module 146. The DSP module 140 may also utilize averaging and median filters to process pose information from the pose estimation module 144, for example, for averaging the movement of specific joints that move together. The combination of filters may reduce noise artifacts between image frames of a gait or balance assessment recording. Digital signal processing may also be used for speech-to-text processing, in combination with the speech processing module 146.

In addition, the DSP module 140 in this embodiment is configured to extract gait/balance and/or cognitive measurements in combination with the measurement extraction module 150, as discussed in more detail below. The DSP module 140 may also be configured to apply digital signal processing methodologies to assign normative scores to certain patient assessment activities, in combination with the normative scoring module 154.

Referring now to FIG. 4B, the pose estimation module 144 is configured to process video data of a patient assessment activity to obtain pose information for the patient. As used herein, “pose information” and “pose feature” are used interchangeably to refer to any information regarding the position and/or orientation of one or more body parts and/or any other suitable information about the pose of a patient during the patient assessment activity.

The pose estimation module 144 comprises one or more pose estimation models 145 that utilize machine learning to predict pose information based on the video data. In this embodiment, the pose estimation model 145 is a deep learning pose estimation model that utilizes a sequence of two-dimensional RGB images as input and outputs three-dimensional points which correspond to specific joints on the human body. An example of a set of joint points is provided in Table 1 below.

TABLE 1
Example Joint Points

| | |
|---|---|
| Left eye | Right eye |
| Left shoulder | Right shoulder |
| Left elbow | Right elbow |
| Left wrist | Right wrist |
| Left hip | Right hip |
| Left knee | Right knee |
| Left ankle | Right ankle |
| Left heel | Right heel |
| Left toe | Right toe |

In some embodiments, the pose estimation model 145 outputs a visual representation of the patient using the joint points. The visual representation may be a skeletal representation or “stick figure” of the patient, illustrating the three-dimensional positions of each of the joint points. In other embodiments, the visual representation may be any other suitable representation.

In some embodiments, the pose estimation model 145 is previously developed model, such as MediaPipe Pose™. In other embodiments, the pose estimation model 145 may be a custom model unique to the system 100. The modular nature of the DAP subsystem 106 allows any of the machine learning models therein to be updated, modified, or replaced as newer technologies become available without change to the core design.

The speech processing module 146 is configured to receive and process audio data of each patient assessment activity to generate speech information. As used herein, “speech information” refers to any information that can be derived from words spoken by or to the patient during the patient assessment activity. Speech information may include “speech features”, “semantic information”, or a combination of both. As used herein, “speech features” refer to indications of the character or quality of the patient's speech including, but not limited to, partial and cut-off words and words that are mispronounced, stuttered, merged, slurred, or mumbled as well as paralanguage elements such as speech variety, confidence, volume, fluency, clarity, pitch, and repetition. As used herein, “semantic information” refers to the meaning behind what is spoken, including the denotation and/or connotation of words and phrases. For example, in a Serial Subtraction test, the semantic information comprises the series of numbers spoken by the patient, regardless of whether the patient's speech is stuttered, mumbled, etc.

The speech processing module 146 may comprise one or more speech-to-text models 147. The speech-to-text models 147 may utilize machine learning to transcribe audio data into transcribed text representing speech information of the patient and any other individuals in the recording of the activity, such as a care provider.

The speech-to-text models 147 may comprise one or more natural language processing (NLP) models. In some embodiments, the NLP model is a large-language model. In some embodiment, the large-language model is multimodal large-language model. The NLP model(s) may encompass the speech-to-text transcription function discussed above or may be in addition thereto. The NLP model(s) may be used for additional processing of the transcribed text generated from the audio data to generate more tangible processed speech information for use by the measurement extraction module 150 discussed below.

The measurement extraction module 150 is configured to extract measurements from the pose information and the speech information as input. As used herein, “measurement” refers to any measurable indication and is inclusive of quantitative and qualitative measurements as well as measuring the presence or absence of a particular feature or action.

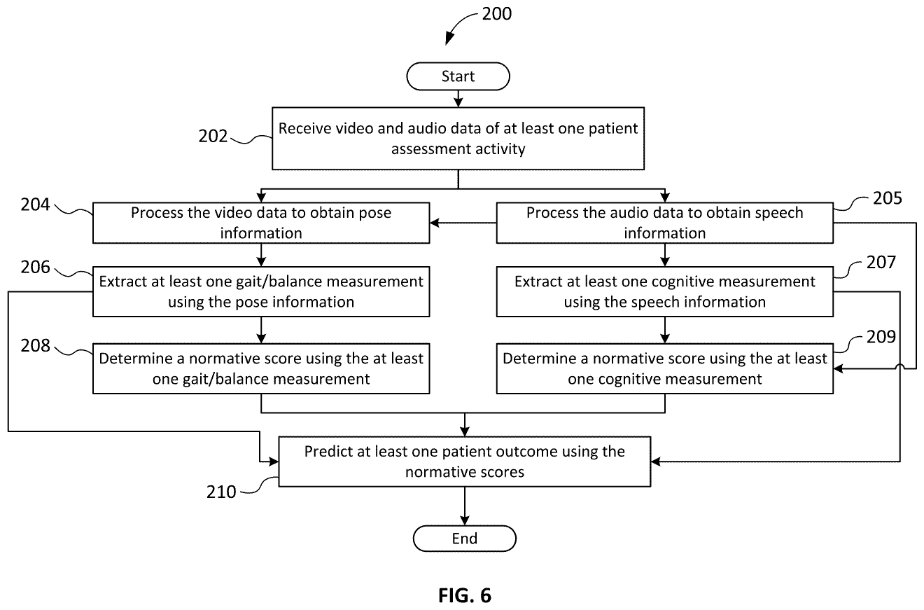

The measurement extraction module 150 may be configured to extract gait/balance measurements from the pose information and cognitive measurements from the speech information. As used herein, “gait/balance measurement” refers to any measurable indication of the movement of one or more body parts of the patient. Examples of gait/balance measurements include but are not limited to: detection of when the patient is in position to begin the assessment; the number of steps taken on each leg during an assessment; step and stride measurements such as stride length, step length, stride width, step width, total distance travelled, and temporal step and stride measurements such as swing time, stance time, stride time, step velocity, and stride velocity; gait cycle phases for each leg; estimation of trunk sway; asymmetry detection during movement; joint angles; and joint velocities. Each of these gait/balance measurements is discussed in more detail below with reference to the method 200 of FIG. 6. In some embodiments, a plurality of gait/balance measurements are extracted from the pose information.

For some gait/balance measurements, the measurement extraction module 150 may use digital signal processing and rule-based models to extract the measurement. For other gait/balance measurements, the measurement extraction module 150 comprises one or more gait/balance measurement extraction models 151 that use machine learning to extract the gait/balance measurement. In some embodiments, the measurement extraction module 150 comprises a plurality of gait/balance measurement extraction models 151. For example, an individual model 151 may be employed for an individual gait/balance measurement. In some embodiments, one or more of the models 151 is a deep learning model.

As used herein, “cognitive measurement” refers to any measurable indication of the cognitive ability of the patient, including measures of thinking, understanding, remembering, paying attention, reasoning, abstraction, and judgement. Examples of cognitive measurements include but are not limited to: measuring hesitation; measuring stuttering and/or cluttering; measuring the ability to understand and follow instructions; measuring the ability to remember and recall words, objects or sentences; measuring orientation; measuring language fluency; and measuring the ability to calculate, problem solve, carry out sequential tasks, or think abstractly. Examples of cognitive measurements are discussed in more detail below with reference to the method 200 of FIG. 6. In some embodiments, a plurality of cognitive measurements are extracted from the speech information. The cognitive measurements may be extracted from speech features, semantic information, or both.

For some cognitive measurements, the measurement extraction module 150 may use digital signal processing and rule-based models to extract the measurement. For other cognitive measurements, the measurement extraction module 150 comprises one or more cognitive measurement extraction models 152 that use machine learning to extract the measurement. In some embodiments, the measurement extraction module 150 comprises a plurality of cognitive measurement extraction models 151. For example, an individual model 152 may be employed for an individual cognitive measurement. In some embodiments, one or more of the models 152 is a large-language model. In some embodiments, the large-language model is a deep learning model. The large-language model may be a multimodal large-language model.

The normative scoring module 154 is configured to determine a normative score for at least one patient assessment activity using the extracted measurements and/or speech information as input. As used herein, a “normative score” for a patient assessment activity refers to a score that indicates the extent to which a patient's performance deviates from an average or “normal” performance for that activity. The “normal” score for that activity may be the stratified based on a number of factors including, for example, healthy vs. a particular disease state, age, sex, etc. Alternatively, the “normal” score may be a “normal” score for a particular patient such as their score from a previous assessment or the average or median score from multiple previous assessments. The normative scoring module 154 may determine a normative score for a gait/balance assessment activity using one or more extracted gait/balance measurements. In some embodiments, the normative scoring module 154 determines normative scores for a plurality of activities and determines a total normative score for the assessment. For example, a score may be assigned for each of the ten activities of the FGA and then a total score determined as the sum of the normative scores for the ten activities. Other gait or balance assessments that can be scored by the normative scoring module 154 include the Dynamic Gait Index (DGI), the “Timed Up and Go” (TUG) test, the Balance Error Scoring System (BESS), and any other suitable gait or balance assessment.

The normative scoring module 154 may also determine a normative score for a cognitive activity using one or more extracted measurements. Alternatively, the normative scoring module 154 may determine some cognitive normative scores directly from speech information. For example, the normative score for a Serial Subtraction test is the number of correct subtractions by a patient in a period of time, which may be determined directly from semantic information in the speech information.

For some assessment activities, the normative scoring module 154 may employ digital signal processing methods to predict a normative score based on the extracted measurements (and/or pose or speech information) and a predetermined ruleset. Digital signal processing may be used for activities that are readily quantifiable, such as counting the number of errors a patient makes in an activity, which is used in certain gait or balance assessments such as the BESS.

For other patient assessment activities, the normative scoring module 154 comprises one or more gait/balance normative scoring models 153 and/or one or more cognitive scoring models 155 that use machine learning to determine the normative score. In some embodiments, one or more of the normative scoring models 153/155 are deep learning neural network models. Machine learning and deep learning models may be used to determine normative scores for patient assessment activities that have previously been more subjective or less precisely quantifiable as they have traditionally been assessed by and within the limits of human observation alone. For example, scores for certain gait/balance activities of the DGI and FGA are based on qualitative observations such as “good gait speed”, “good balance”, etc. that would normally be made by a trained expert (e.g., care provider). Machine learning and deep learning models may be used to determine such scores with much more precision than crude observational measures and qualitative judgements currently made by trained clinicians.

In some embodiments, the cognitive normative scoring models 155 may further comprise multi-modal large-language models for scoring of certain cognitive assessments, such as questions relating to abstractions.

In some embodiments, the measurement extraction module 150 and/or the normative scoring module 154 may also determine a “cost” factor for dual-task assessments. For a gait/balance assessment, the cost factor is the difference between the measurement or score of a dual-task gait/balance assessment and that of an individual gait/balance assessment. Similarly, for a cognitive assessment, the cost factor is the difference between the measurement or score of a dual-task cognitive assessment and that of an individual cognitive assessment.

The outcome prediction module 156 is configured to predict at least one patient outcome using one or more measurements from the measurement extraction module 150 and/or one or more normative scores from the normative scoring module 154 as input. As used herein, “patient outcome” is inclusive of any potential risks for the patient, such as risk of falling, as well as predictions of future cognition, gait and/or balance, diagnoses of health conditions, and predictions of changes to the patient's cognition, gait and/or balance over time.

The outcome prediction module 156 may comprise one or more outcome prediction models 157 that use machine learning to generate the prediction. In some embodiments, one or more of the outcome prediction models 157 utilizes deep learning methodologies including, but not limited to, gradient boosting models and deep learning neural networks to generate the prediction. The outcome prediction models 157 may utilize the output of any of the previous models including pose information from pose estimation module 144, speech information from the speech processing module 146, extracted measurements from the measurement extraction module 150, and/or normative score(s) from the normative scoring module 154 to generate the prediction to generate the prediction, alone or in combination. The outcome prediction models 157 may also use the cost factors from dual-task assessments as input. In some embodiments, the outcome prediction models 157 uses a combination of at least one gait/balance measurement and/or gait/balance normative score and at least one cognitive measurement and/or cognitive normative score to generate the prediction. This combination can provide more accurate predictions of patient outcomes than just gait/balance measurements or normative scores alone. In some embodiments, the outcome prediction models 157 may initially use normative scores from gait/balance assessments as input but, as the models 157 are trained on more gait/balance measurements over time, the normative scores may be omitted and the models 157 may generate predictions directly from gait/balance measurements such as step and stride measurements.

In some embodiments, the outcome prediction models 157 also utilize extracted measurements and/or normative scores obtained from different patient assessments performed at different times to predict changes in the patient's condition over time, such as deterioration of a health condition or response to a new treatment regimen. In some embodiments, the models 157 may also use patient demographics such as the patient's age, biological sex, etc. in combination with the model output data to make the predictions.

In some embodiments, the outcome prediction models 157 may comprise separate models for gait/balance predictions and cognitive predictions. In other embodiments, the gait/balance and cognitive prediction models are developed together and combined into a multimodal system.

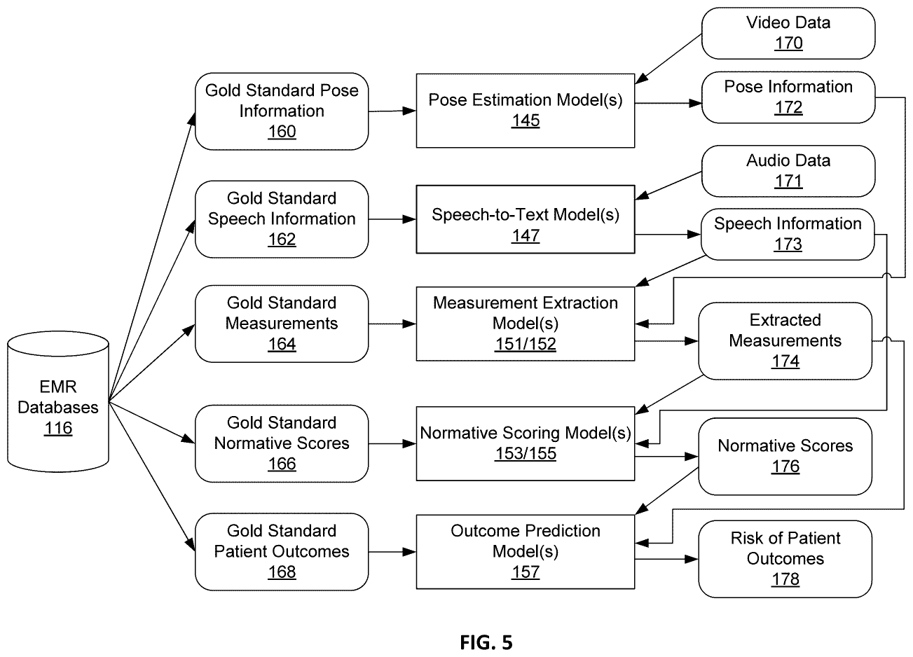

FIG. 5 is a schematic of an example training structure for the machine learning models of FIG. 4B.

The pose estimation model(s) 145 may be trained using a gold standard pose information dataset 160. The phrase “gold standard” in this context refers to a dataset that is obtained using benchmark or conventional means or using the best available means under reasonable conditions. Gold standard data is used as the training data for the machine learning models of the system 100.

In some embodiments, the pose information dataset 160 comprises pose features data obtained from one or more “gold standard” devices that physically attach to a patient or with which the patient otherwise physically interacts with. A gold standard device may also be referred to as a “physical sensor”. For example, the physical sensor may comprise at least one of: a marker-based motion capture system, one or more wearable sensors, and a pressure sensor walkway or treadmill. The data from these devices (“sensor data”) may be collected alongside video data while the patient performs at least one gait or balance assessment activity to form the gold standard pose features. Data from a plurality of gait and balance assessment activities can thereby form the dataset 160. The gait/balance assessment activity may be performed individually or as part of a dual-task assessment. In other embodiments, the gold standard pose information dataset 160 may comprise any other suitable dataset, including one or more public datasets if available. The dataset 160 may be stored in the EMR databases 116, for example, in the assessment database 130. The trained pose estimation model(s) 145 may then be used to process video data 170 of patient gait or balance assessment activities to generate pose information 172. The model(s) 145 may be further refined and updated as more video data 170 is uploaded and processed.

The speech-to-text models 147 may be trained using a gold standard speech information dataset 162. The gold standard speech information dataset 162 may comprise a specialized cognitive assessment dataset in addition to publicly available speech-to-text datasets. The speech information dataset 162 may include both speech features and semantic information data. The cognitive assessment dataset may be collected during cognitive assessments performed alongside gait/balance assessments, both individually and as part of a dual-task assessment. Audio data may be recorded during the assessment and may be transcribed, assessed and labeled by trained experts such as clinicians to form the gold standard speech information.

The trained speech-to-text models 147 may then be used to process audio data 171 of patient cognitive assessment activities to generate speech information 173 in the form of transcribed text. The models 147 may be further refined and updated as more audio data 171 is uploaded and processed.

The gait/balance and cognitive measurement extraction model(s) 151 and 152 may be trained using a gold standard measurements dataset 164. The gold standard measurements dataset 164 includes datasets for both gait/balance measurements and cognitive measurements. The measurements may be collected from both dual-task and individual assessments and may be tagged or labeled as such to facilitate training. The gait/balance measurements dataset may comprise gait/balance measurement data obtained using one or more of the same devices described above for the pose information dataset 160. In some embodiments, the gait/balance data is collected at the same time as the pose information dataset 160 as a patient performs at least one gait or balance assessment activity. Alternatively, or additionally, at least some of the gait/balance measurements may comprise gait/balance measurements extracted from the pose features in the pose information dataset 160. Non-limiting examples of gait/balance measurements in the gait/balance measurement dataset include: step and stride measurements such as stride length, step length, stride width, step width, total distance travelled, and temporal measurements such as swing time, stance time, stride time, step velocity, and stride velocity; gait cycle phase measurements; estimation of trunk sway; asymmetry detection during movement; joint angles; and joint velocities.

The cognitive measurement dataset may comprise measurements of various speech features including, for example, variety, volume, fluency, clarity, pitch, repetition, stuttering, and hesitation in speech. In some embodiments, the measurements are collected by trained experts such as clinicians during cognitive assessments. In some embodiments, the cognitive measurements are collected at the same time as the speech information for the gold standard speech information dataset 162. For example, a clinician may identify and count instances of stuttering. Alternatively, or additionally, at least some of the cognitive measurements may be extracted from the speech information in the gold standard speech information dataset 162.

The gold standard measurements dataset 164 may be stored in the EMR databases 116, for example, in the assessment database 130. In some embodiments, the pose information dataset 160, the gold standard speech information dataset 162, and the gold standard measurements dataset 164 are combined into a single dataset.

In other embodiments, the gold standard measurements dataset 164 may further comprise any other suitable dataset, including one or more public datasets if available.

The trained measurement extraction models 151/152 may then be used to extract one or more extracted measurements 174 from the pose information 172 and/or the speech information 173. The models 151/152 may be further refined and updated as more pose information 172 and speech information 173 are obtained from patient video data 170 and audio data 171 and more measurements 164 are extracted.

The gait/balance and cognitive normative scoring model(s) 153 and 155 may be trained using a gold standard normative scores dataset 166. The normative scores dataset 166 may comprise normative scores assigned to patient gait/balance assessment activities and cognitive activities by one or more trained experts, such as care providers, academic researchers, or any other person with sufficient expertise to assign a normative score to a gait/balance or cognitive assessment activity. The normative scores dataset 166 may include scores for individual gait/balance or cognitive activities as well as scores for dual-task assessment activities. In some embodiments, scores are tagged as being either individual or dual-task scores to facilitate training.

In some embodiments, the normative scores are collected alongside the pose and speech information datasets 160, 162 and the measurements dataset 164. For example, the trained expert may assign a score to a given gait/balance or cognitive assessment activity as the activity is performed and the pose information, speech information, and measurements are collected. Collecting the pose and speech information, measurements, and normative scores during the same assessments may improve the efficiency of the model training and consistency between different models.

Alternatively, or additionally, the normative scores dataset 166 may comprise normative scores that are assigned by care provider users of the system 100 who review video data 170 and/or audio data 171 of one or more gait/balance or cognitive assessment activities and enter their score via the user interface application 111. In this embodiment, the dataset 166 continues to grow as more care providers use the system 100.

In some embodiments, the gold standard normative scores dataset 166 may further comprise any other suitable dataset, including one or more public datasets if available.

The gold standard normative scores dataset 166 may be stored in the EMR databases 116, for example, in the assessment database 130.

The trained gait/balance and cognitive normative scoring model(s) 153 and 155 may then be used to predict normative scores 176 for one or more gait/balance or cognitive assessment activities using the extracted measurements 174 and/or speech or pose information. The normative scoring model(s) 155 may be further refined and updated as more pose information 172, speech information 173, and extracted measurements 174 are available.

The outcome prediction model(s) 157 may be trained using a gold standard patient outcome dataset 168. The gold standard patient outcome dataset 168 may comprise data regarding outcomes in association with cognitive, gait, and balance assessment results, including measurements and scores from both individual and dual-task assessments as well as dual-task cost factors. The patient outcomes may be patient-reported outcomes from patient users of the system 100. For example, patients may report the number of falls they've experienced over a given time period. By using patient-reported outcomes, the system 100 does not need to process large patient history datasets to obtain the relevant data but rather receives outcome data directly associated with the assessment measurements and scores. Alternatively, or additionally, the patient outcomes may be reported by caregiver users of the system 100. The patient outcome dataset 168 may thereby grow as more patients and caregivers use the system. In some embodiments, the patient outcome dataset 168 also includes patient demographics such as the patient's age, biological sex, etc. Regardless of the source, the data collected regarding a particular patient may first be de-identified before being included in the patient outcome dataset 168. In other embodiments, the gold standard patient outcomes dataset 168 may comprise any other suitable dataset, including one or more public, de-identified datasets if available.

In some embodiments, the outcome prediction models 157 may comprise separate models for gait/balance predictions and cognitive predictions, trained independently on gait/balance or cognitive training data, respectively. In some embodiments, the gait/balance and cognitive prediction models are developed in parallel and combined into a multimodal system. The dataset 168 may be stored in the EMR databases 116, for example, in the assessment database 130.

The trained outcome prediction models 157 may then be used to predict one or more patient outcomes 178 using one or more measurements and/or normative scores 176 for that patient. The outcome prediction models 157 may also use pose information 172, speech information 173, and/or any other output from any of the models for generating the prediction. The model(s) 157 may be further refined and updated as more normative scores 176 and extracted measurements 174 are available and more predictions are made.

FIG. 6 is a flowchart of an example method 200 for assessing a patient, according to some embodiments. The method 200 is discussed with reference to the system 100. Although the blocks in the method 200 are shown sequentially in the flowchart in FIG. 6, it will be understood that some steps may be done simultaneously or in a different order. For example, the data processing steps at blocks 204, 205 are shown in FIG. 6 as occurring after the video and audio data is received at block 202 but some processing may occur as the video and audio data is being recorded in order to provide real-time feedback or results.

Prior to the steps of the method 200, a user may record video and audio data of a patient performing at least one gait or balance assessment activity and/or at least one cognitive assessment activity via the video capture device 115 and audio capture device 117 of the user device 102. The gait/balance assessment activity may be performed at the same time as the cognitive assessment activity (dual-tasking) or sequentially. Alternatively, the gait/balance assessment activity can be performed at one time and the cognitive assessment activity could be performed at a different time and two separate recordings can be generated.

In some embodiments, the gait or balance assessment activity is an activity in a standard gait or balance assessment such as the FGA, DGI, TUG, or BESS assessments. In some embodiments, video/audio data includes all of the activities of the standard gait or balance assessment, such as all ten activities of the FGA. In other embodiments, the gait or balance assessment activity may be any other suitable activity, or combination of activities, relevant to assessing a patient's gait, balance, etc.

In some embodiments, the cognitive assessment activity may be a standard cognitive assessment activity such as Serial Subtractions or other activities within a MoCA or Mini Mental Status Exam (MMSE). In other embodiments, the cognitive assessment activity may be any other activity relevant to a patient's cognitive ability. In some embodiments, the cognitive assessment activity may not be a separate activity but rather may comprise words spoken by the patient during the gait/balance assessment activity and/or the ability of the patient to understand and follow the gait/balance assessment activity instructions.

In some embodiments, the user is the patient performing a self-assessment. In other embodiments, the user and the patient may be different individuals. For example, the user may be the patient's care provider or another individual assisting the patient in completing the assessment. In some embodiments, the system 100 prompts the user/patient, via the user interface application 111, to perform an assessment activity and may provide instructions for performing the activity. In some embodiments, the system 100 provides feedback to the patient while they complete the assessment activity, for example, through visual and/or audio cues as discussed above.

At block 202, the system 100 receives video and audio data of at least one gait or balance assessment activity and/or at least one cognitive assessment activity performed by the patient. The video and audio data may be received from the user device 102 via the communication network 103. In some embodiments, the video and audio data are received together as a single recording. In other embodiments, the video and audio data may be received separately. In some embodiments, the system 100 may synchronize the video and audio data if it is not already synchronized.

In some embodiments, the recording may include extraneous content in addition to the assessment activity itself, such as the patient getting ready for the assessment activity (e.g., setting up the user device 102, getting into position, etc.). The extraneous content may also include dialogue between the patient and the care provider (or other individual assisting the patient in the assessment), such as greetings, personal conversations, farewells, etc.

At block 204, the video data is processed to obtain pose information for the patient. The processing step may be done by the pose estimation module 144 using at least one pose estimation model 145. In some embodiments, processing the video data comprises assigning a plurality of three-dimensional joint points to the two-dimensional video data of the patient. The joint points may be the joint points provided above in Table 1 or any other suitable set of joint points. In some embodiments, processing the video data further comprises generating a visual representation of the patient, such as a “stick figure” or skeletal representation.

In some embodiments, the video data and/or the pose information are filtered prior to the steps of block 206 to reduce noise. Filtering may be performed by the digital signal processing module 140. For example, combinations of Savitsky-Golay, Kaiser-Bessel, and Weiner filters may be used to reduce the effect of noise on output from the pose estimation model(s) 145. In some embodiments, filtering may further comprise averaging the movement of joints that move similarly together such as by applying average or median filters. For example, the heel and the ankle on the same foot may be averaged as they move similarly when a step is taken.

At block 205, the system 100 processes audio data to obtain speech information. The processing step may use a combination of the speech-to-text models 147 as well as digital signal processing and other software algorithms.

In some embodiments, processing the audio data comprises converting (transcribing) sounds from the audio data to transcribed text via the speech-to-text models 147. The transcribed text represents speech information spoken during the patient assessment. In some embodiments, the system 100 assigns timestamps to the transcribed text representing the time at which each word or other speech feature is spoken. In some embodiments, the system 100 assigns speaker labels to the transcribed text for differentiating between the patient and care provider (and any other individuals) in the recording.

The system 100 may then further process and refine the transcribed text using natural language processing to generate a more tangible refined transcribed text. For example, the system 100 may use NLP models to refine the timestamps and/or speaker labels for improved accuracy and precision. In some embodiments, the system 100 identifies specific keywords or phrases (“speech cues”) indicating the start or end of a particular portion of the transcribed text and assigns a timestamp for each speech cue. In some embodiments, the system 100 may classify or categorize different portions of the transcribed text, such as a greetings portion, an instruction-giving portion, any instruction-clarification portions, one or more assessment portions, and a conclusion portion. Portions of the transcribed text may also be categorized as questions and answers from either the care provider or patient. The system 100 may also use NLP models to identify verbal corrections made during the assessment activity and critical key words mentioned at any time during the patient assessment.

In some embodiments, the system 100 may generate a visual representation of the transcribed text that can be displayed to the user via the user interface application 111 and/or stored for future reference.

In some embodiments, the system 100 uses the transcribed text generated at block 205 to further process the video data at block 204. In some embodiments, this further processing comprises assigning one or more timepoints to the video data based on one or more timestamps of the transcribed text. For example, the system 100 may assign a timepoint to the video data indicating the point at which the patient assessment activity actually starts. As noted above, the recording may start before the actual assessment activity and may include extraneous content. The system 100 may use the NLP models discussed above to identify a speech cue indicating the actual start of the assessment activity such as a prompt from the care provider and/or confirmation words from the patient indicating they are ready. The system 100 may thereby label a timestamp of a particular speech cue as an “activity start” timestamp. The system 100 may then assign an “activity start timepoint” to the video data based on the activity start timestamp in the transcribed text.

Similarly, the system 100 may also assign an “activity end timepoint” to the video data based on a corresponding timestamp in the transcribed text at which a speech cue indicates that the care provider instructed the patient to stop or the patient indicated that they were done.

In some embodiments, the system 100 then crops the video data based on the activity start and/or end timepoints. For example, the system 100 may crop the video between the activity start and end timepoints to remove any preceding or subsequent video. By cropping the video data to just the patient assessment activity, the system 100 may only generate pose information for the relevant portions of the video data, and thus the system 100 may use less processing power (fewer instructions) than if the full recording was processed.

In embodiments in which the video data contains both a gait/balance assessment activity and a cognitive assessment activity (or more than one gait/balance assessment activity), the system 100 may also assign timepoints to the video data indicating when each individual activity starts and ends based on corresponding timestamps of speech cues in the transcribed text. The system 100 may thereby define individual segments of the video data. In some embodiments, the system 100 crops the video data into one or more individual segments based on individual activity start and end timepoints. The individual segments may then be provided to different measurement extraction models 151/152 at block 206 below such that specific measurements are extracted from each individual segment. Alternatively, the system 100 may label or tag one or more segments in the video data and each measurement extraction model 151/152 may be provided with the full video recording but may be configured to only extract certain measurements from one or more labeled segments.

At block 206, at least one gait/balance measurement is extracted from the pose information. The extraction step may be done using a combination of digital signal processing and at least one gait/balance measurement extraction model 151 of the measurement extraction module 150.

In some embodiments, one of the gait/balance measurements may comprise detection of when the patient is in position to begin the assessment activity. For example, the patient may be ready for the assessment when they are standing in double support (i.e., with both feet in contact with the ground). This measurement may be extracted from the pose information by calculating the position and motion of the patient's feet relative to the rest of their body. Alternatively, or additionally, the patient may indicate their readiness by making a gesture to the video capture device 115 such as a thumbs-up. One of the gait/balance extraction models 151 may be a deep learning model designed for gesture recognition that recognizes the thumbs-up gesture as an indication that the patient is ready to start the assessment activity.

In some embodiments, one of the gait/balance measurements comprises the number of steps taken on each leg during an assessment. The number of steps may be extracted from the pose information by: calculating the velocity of each foot during the assessment activity with a numerical differentiation; determining the locations of peaks and their prominences; and filtering the high prominence peaks to become the number of steps. In other embodiments, the number of steps may be extracted from the pose information by one of the machine learning models 151 or any other suitable method.

In some embodiments, the gait/balance measurements include one or more step and stride measurements such as stride length, step length, stride width, step width, total distance travelled, and temporal step and stride measurements such as swing time, stance time, stride time, step velocity, and stride velocity. These measurements are calculated by filtering to remove noise in the pose information and then using vector calculus to determine various relevant velocity and acceleration vectors. Depending on the measurement, additional digital signals processing filtering may occur to further reduce noise in these vectors.

In some embodiments, one of the gait/balance measurements comprises classification of gait cycle phases for each leg. This measurement may be extracted by: calculating the velocity of each foot during the assessment activity with numerical differentiation; calculating the power spectral density to determine a noise floor; and predicting frames where each leg is in a stance or swing phase depending on the calculated metrics. The frame prediction may be performed by one of the machine learning models 151, in some embodiments. In other embodiments, the gait cycle phases may be classified by any other suitable method. In other embodiments, any other suitable gait cycle phase metrics may be extracted from the pose information.

In some embodiments, one of the gait/balance measurements comprises an estimation of trunk sway. Trunk sway may be estimated using the pose features by calculating the vector determining tilt of the patient's shoulder joints relative to their hip joints. In other embodiments, trunk sway may be estimated using any other suitable method.

In some embodiments, one of the gait/balance measurements comprises asymmetry detection during movement. This measurement may be extracted from the pose information by comparing left-and right-sided movements of the patient and calculating various statistical measures (e.g., average, standard deviation) to detect asymmetry. In other embodiments, asymmetry may be detected using any other suitable method.

In some embodiments, one of the gait/balance measurements comprises joint mobility calculations, such as joint angles and/or joint velocities. These measurements may be extracted from the pose information by the gait/balance measurement extraction models 151 using the pose estimation model 145 outputs as well as spatial measurements regarding patient assessment setups. The system 100 may estimate the spatial measurements from the video data. Alternatively, the spatial measurements may be inputted by the user via the user interface application 111. In some embodiments, an initial estimate of the spatial measurements is extracted from the video data and then the measurements are refined using user input (e.g., the user's height, how far they walked during the assessment, etc.). In other embodiments, joint mobility calculations may be performed using any other suitable method.

At block 207, at least one cognitive measurement is extracted from the speech information. The extraction step may be done using a combination of digital signal processing and at least one cognitive measurement extraction model 152 of the measurement extraction module 150.

In some embodiments, one of the cognitive measurements comprises measuring hesitation or pauses in the patient's speech. Hesitation may be measured by calculating the time between timestamps of speech features such as the time between spoken words, the time between a question asked by the care provider (or the user interface 112) and the answer provided by the patient, and/or the time between provided answers. In other embodiments, hesitation may be measured by any other suitable means.

In some embodiments, one of the cognitive measurements comprises measuring stuttering and/or cluttering in the patient's speech. This measurement may comprise quantifying difficulty producing words, counting words that are merged, slurred, or mumbled, and/or counting the number of cut-off parts of words. In other embodiments, stuttering and/or cluttering may be measured by any other suitable means.

In some embodiments, one of the cognitive measurements comprises measuring the ability to understand and follow instructions. This measurement may comprise counting the number of clarifications that the care provider gives the patient before or during the activity and/or detecting at least one acknowledgement of understanding from the patient (e.g. saying an affirmation like “yes”) within a certain time period following the care provider giving an instruction. For self-assessments, the number of visual or audio cues or feedback from the user interface 112 may be counted. In some embodiments, the cognitive measurements are extracted from the speech information from audio data of a cognitive assessment activity. However, certain cognitive measurements may be extracted from other portions of the audio data, including the audio data for a gait/balance assessment activity. For example, the ability of the patient to understand instructions may be assessed during the gait/balance assessment activity instead of, or in addition to, the cognitive assessment activity. Moreover, measurements relating to speech quality such as hesitation, stuttering, etc., and/or the patient's overall affect, may be derived from speech information captured at any time in the recording, including before, during, or after a particular activity. In this manner, video and audio data of a single gait/balance assessment could be used to obtain both gait/balance and cognitive measurements.

Of note, although FIG. 6 shows the cognitive measurements at block 207 as using the speech information from the processed audio data at block 205, not all measurements may require transcribed text of the audio data and some measurements may be derived directly from the audio data including, for example, some measurements relating to speech feature like volume, pitch, etc.

In some embodiments, the system 100 may extract one or more gait/balance or cognitive measurements using both the pose information and the speech information. For example, many of the gait/balance measurements discussed above are timed measurements in which certain features are counted or measured over a set period of time. Without the speech information, the activity start timepoint may be assigned based on when the patient starts moving (the “movement start point”). However, with speech information, the system 100 may assign the activity start timepoint based on a corresponding timestamp in the transcribed text in which a speech cue indicates that the activity has started, as discussed above. The assigned activity start timepoint based on the transcribed text may be more accurate than a timepoint assigned based solely on patient movement as there may be a lag between when the patient is told to start and when they actually start moving. In addition, the time lag between the activity start timepoint based on the speech information and the movement start point based on the pose information may represent an additional gait/balance or cognitive measurement as a longer time lag may indicate that the patient has difficultly initiating movement or understanding the instructions.

Similarly, the system 100 may use the speech information to determine a more accurate activity end timepoint. Without speech information, the gait/balance activity may be measured until the point at with the patient stops the movement (the “movement end point”). However, speech cues may indicate if the care provider has prompted the patient to keep going or if the patient ignored an instruction to stop.

It will be understood that there are many other possible gait/balance and cognitive measurements that can be extracted from the pose information and speech information and embodiments are not limited to only the specific measurements described above.

At block 208, a normative score is determined for at least one gait or balance assessment activity using the gait/balance measurements. In some embodiments, a respective score is determined for each activity of a standard gait or balance assessment, such as each of the ten activities of the FGA as one example. The gait/balance normative scores may be determined using a combination of digital signal processing and at least one gait/balance normative scoring model 153 of the normative scoring module 154. As discussed above, the system 100 may use digital signal processing methods to predict a normative score for more quantitative assessment activities based on the extracted gait/balance measurements and a predetermined ruleset. For example, the BESS contains an assessment activity that involves counting the number of times a patient may stumble or remove their hands from their hips when standing on one leg. Each error adds one point to their normative score for the BESS. Digital signal processing may be used to calculate the velocities of the patient's feet and significant motion may be counted as an error. Similarly, if the spatial relation of the patient's wrist joint and their hip joints indicates they have removed their hands from their hips, then another error is counted. Following this ruleset and using the extracted gait/balance features, the normative score for the BESS can be calculated.

For other gait or balance assessment activities that are more qualitative, the system 100 may use one or more gait/balance normative scoring models 153 to determine the normative score. For example, the score for the FGA “gait level surface” is a normative score from 0 to 3, with 3 indicating normal performance and 0 indicating severe impairment. The score is based on qualitative observations of gait such as “good gait speed”, “good balance”, etc. that would normally be made by a trained expert (e.g., care provider). The normative scoring models 153 may use machine learning and/or deep learning methodologies to assign a normative score based on the extracted gait/balance measurements. In some embodiments, the appropriate model 153 may determine quantitative values in place of the qualitative observations normally used in the assessment. For example, rather than indicating “good gait speed”, the model 153 may calculate the patient's gait speed and determine if the gait speed is within the normal range.

In some embodiments, the method 200 further comprises determining a total gait or balance performance value for the patient. The total performance value may be based on the normative scores for individual assessment activities and may also be based on certain extracted measurements from block 206. In some embodiments, some scores and/or measurements may be weighted more than others in determining the total performance value. In some embodiments, two or more different weighted total performance values may be determined such as one value for use in the subsequent model predictions at block 210 and a different value for display to a care provider and/or patient. The total performance value for model predictions may weigh certain factors more heavily based on their predictive value, whereas the total performance value for the care provider may be weighted to indicate how well the patient performed in particular aspects of the assessment. For example, an aggregated, weighted performance value may be calculated for all of the gait (and not balance) aspects of the assessment and that value may be displayed to the care provider to indicate the patient's overall gait performance. A separate performance value could be determined for balance.

In some embodiments, the method 200 may further comprise determining a cost factor for a gait/balance assessment performed as part of a dual-task assessment. The cost factor may be determined by calculating the difference between a normative score (and/or measurement) from the gait/balance assessment activity performed as part of the dual-task assessment compared to the same assessment activity performed independently. The dual-task assessment and individual assessment may be included in the same video/audio recording (which may be segmented using speech cues as discussed above) or may be in separate recordings from which measurements and scores are extracted and compared.

At block 209, a normative score is determined for at least one cognitive assessment activity. In some embodiments, a respective score is determined for each activity of a standard cognitive assessment, such as the MoCA.

The cognitive normative scores may be determined using a combination of digital signal processing and at least one cognitive normative scoring model 155 of the normative scoring module 154. The normative score may be determined based on one or more extracted measurements or may be extracted directly from semantic information in the speech information. For example, the score for the Serial Subtractions test may be directly derived from semantic information in the speech information by determining the number of correct subtractions in a set period of time.

As discussed above, the system 100 may use digital signal processing methods to predict a normative score for more quantitative assessment activities based on the speech information or extracted cognitive measurements and a predetermined ruleset. For other more abstract cognitive assessment activities, the system 100 may apply one or more of the cognitive normative scoring models 155, including large-language models. In some embodiments, the method 200 may further comprise determining a total cognitive performance value for the patient. The total cognitive performance value may be based on normative scores and/or measurements from multiple assessment activities. In some embodiments, more than one weighted cognitive performance value may be determined in a similar manner as discussed above with respect to the total gait or balance performance value. In some embodiments, the method 200 further comprises determining a cost factor for a cognitive assessment performed as part of a dual-task assessment in a similar manner to that discussed above for gait/balance assessments.

At block 210, at least one patient outcome is predicted using at least one measurement and/or normative score. The patient outcomes may be predicted using at least one outcome prediction model 157 of the outcome prediction module 156.

In some embodiments, the patient outcome is a risk prediction, such as the risk of the patient falling or otherwise injuring themselves. Alternatively, or additionally, the patient outcome may be a long-term prediction of the patient's health, such as predicting early signs of a health condition that involves cognition, gait, and/or balance issues. In some embodiments, the outcome prediction module 156 may also generate a predicted diagnosis. The outcome prediction module 156 may also predict improvements or deterioration of a cognitive and/or neuromuscular health condition or disease state over time, including response to treatment, adverse events, etc. Non-limiting examples of such health conditions or disease states include aging, neurologic or neurodegenerative conditions (e.g., Alzheimer's dementia, multiple sclerosis, Parkinson's), neuromuscular conditions (e.g., Guillain-Barre syndrome, peripheral neuropathies), concussions or traumatic brain injuries, musculoskeletal injuries (e.g., from falls) or post-operative states (e.g., hip replacement).

In some embodiments, the outcome prediction models 157 use a combination of measurements and/or normative scores and patient demographics (e.g., patient age, biological sex, etc.) to generate the prediction. The models 157 may also incorporate the output from some or all of the other preceding models, including e pose information and speech information. In some embodiments, the outcome prediction models 157 use a combination of at least one gait/balance measurement and/or gait/balance normative score and at least one cognitive measurement and/or cognitive normative score to generate the prediction. The outcome prediction models 157 may also use cost factors from dual-task assessments as input. In some embodiments, the outcome prediction models 157 use respective normative scores and/or extracted measurements from patient assessment activities completed at different times, for example, to predict long-term patient outcomes such as changes to cognition and/or gait or balance over time.

In some embodiments, the patient outcome may be expressed on a linear scale. For example, fall risk may be expressed as a number between one and ten, or as a percentage, etc. This allows for more precise, accurate and reliable predictions compared to current assessments that merely classify patients into broad risk stratifications such as low-, medium-, and high-risk.

The method 200 may further comprise steps for generating and managing patient records via the EMR subsystem 104. In some embodiments, the method 200 further comprises storing video and audio data, extracted measurements, normative scores, and patient outcome predictions in a patient record, which may be accessed by users depending on their permission level. The method 200 may further comprise generating a log when the patient record is accessed via the user interface application 111 and/or when a change to the record is made.

In some embodiments, the method 200 further comprises pushing or transmitting a notification to a user, via the user interface application 111. For example, the notification may prompt the patient to perform a self-assessment or prompt the patient to make an appointment with their care provider. Notifications may also be generated to users in response to an assessment-related event, such as when a new assessment recording is uploaded or when a new patient outcome prediction is generated. Additional examples of notifications are discussed above with respect to the notifications web API 122.

In some embodiments, the method 200 may further comprise prompting the user, via the user interface application 111, to perform one or more therapeutic or rehabilitative activities, such as performing certain exercises to improve their gait or balance. Other variations of the method 200 are possible. As discussed above, as the outcome prediction models 157 are trained on more measurements and normative score data, the normative scores may be omitted and predictions may be made directly from one or more extracted measurements. In addition, although FIG. 6 shows steps for both gait/balance and cognitive assessments, some predictions may be made from just gait/balance measurements and/or normative scores and other predictions may be made from just cognitive measurements and/or normative scores and the other steps may be omitted in those cases.

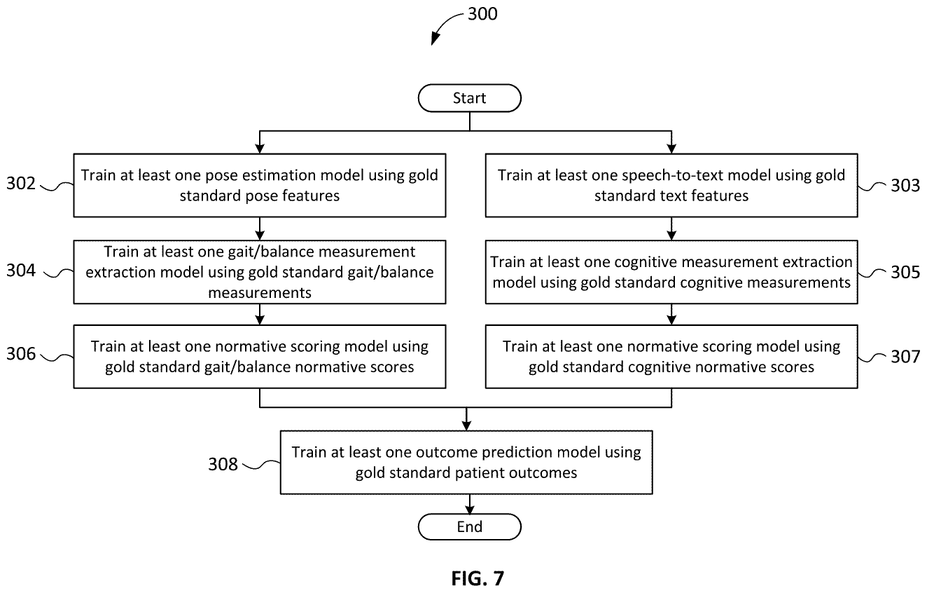

FIG. 7 is a flowchart of an example method 300 for training the machine learning models of the system 100, used in the method 200.

At block 302, at least one pose estimation model 145 is trained using the gold standard pose information dataset 160. In some embodiments, more than one machine learning model may be trained for pose estimation, either on the same dataset or on a different dataset.

In some embodiments, the method 300 further comprises collected gold standard pose information data for the pose information dataset 160. The gold standard pose information data may comprise sensor data from patients performing at least one gait or balance assessment activity using a physical sensor such as a marker-based motion capture system, wearables, a pressure sensor walkway or treadmill, etc.

Each pose estimation model 145 may be trained by splitting the gold standard pose information dataset 160 into train-validation-test portions. The model may be trained on the training portion, fine-tuned on the validation portion, and evaluated on the test portion. In other embodiments, each pose estimation model 145 may be trained by any other suitable machine learning or deep learning model training methodologies. In some embodiments, the method 300 further comprises comparing the trained pose estimation model(s) 145 with a standard metric, such as mean Average Precision (mAP).

At block 303, at least one speech-to-text model 147 is trained using gold standard speech information 162. In some embodiments, one or more of the speech-to-text models 147 are natural language processing models, large-language models, and/or multimodal large-language models.

In some embodiments, the method 300 further comprises collecting gold standard speech information data for the dataset 162. Gold standard speech information data may comprise audio data from a plurality of patient cognitive assessment activities along with transcribed text that has been transcribed and labeled by a trained expert such as a clinician. The cognitive assessment activities may be performed alongside the gait/balance assessment activities used to collect gold standard pose information data as discussed above, both individually and as a dual-task assessment.

Each speech-to-text model 147 may be trained using any suitable machine

learning or deep learning model training methodologies. In some embodiments, the method 300 further comprises comparing the trained speech-to-text models 147 with a standard metric, such as Diarization Error Rate and Word Error Rate.

At block 304, at least one gait/balance measurement extraction model 151 is trained using gait/balance measurement data in the gold standard measurement dataset 164. In some embodiments, more than one machine learning model may be trained for gait/balance measurement extraction, either on the same dataset or on a different dataset. For example, different models may be trained to extract different gait/balance measurements.

At block 305, at least one cognitive measurement extraction model 152 is trained using cognitive measurement data in the gold standard measurement dataset 164. In some embodiments, more than one machine learning model may be trained for cognitive measurement extraction, either on the same dataset or on a different dataset. For example, different models may be trained to extract different cognitive measurements.

In some embodiments, the method 300 further comprises collecting gold standard gait/balance and/or cognitive measurement data for the dataset 164. Gold standard gait/balance measurement data may comprise sensor data collected from a plurality of gait/balance assessment activities using physical sensors such as a marker-based motion capture system. In some embodiments, the gold standard pose features and the gold standard gait/balance measurements are collected at the same time using the same device. Alternatively, or additionally, gold standard gait/balance measurements may be extracted from the gold standard pose features. Gait/balance measurement data may be collected during both individual assessments and dual-task assessments.

Gold standard cognitive measurements may include measurements made by a trained expert during a cognitive assessment activity, such as counts of hesitation, stuttering, and/or cluttering in speech. Cognitive measurements may be collected at the same time as the speech feature data for the speech information dataset 162 and may be based on the same transcribed and labeled text. Cognitive measurement data may be collected during both individual assessments and dual-task assessments.

The gait/balance measurement extraction model(s) 151 and the cognitive measurement extraction models 152 may be trained using train-validation-test techniques or using any other suitable machine learning or deep learning techniques.

At block 306, at least one gait/balance normative scoring model 153 is trained for normative score determination using the gold standard normative score dataset 166. In some embodiments, more than one machine learning model may be trained for normative score determination, either on the same dataset or on a different dataset. For example, different models may be trained to score different assessment activities.

In some embodiments, the method 300 further comprises collecting gold standard gait/balance normative scores for the dataset 166. The gold standard normative scores may comprise scores assigned by trained experts to patients performing at least one gait/balance assessment activity. In some embodiments, a first dataset of normative scores is collected alongside the pose information dataset 160 and the gait/balance measurement dataset 164, that is, the trained expert assigns a score to a given gait or balance assessment activity as the activity is performed and data is collected by the physical sensors. A second dataset may be collected from care provider users using the system 100 who enter their own scores via the user interface application 111. In some embodiments, the first dataset may be used to initially train the models 153 (e.g., via a train-validation-test split) and the second dataset may be used to monitor model drift or performance as more data is collected. When the second dataset reaches a suitable size, another train-validation-test split of this data may be used to compare different trained models 153. The second dataset may be continually adjusted to adapt to changing real-world contexts to maintain accuracy and precision of the models 153.

At block 309, at least one cognitive normative scoring model 155 is trained for normative score determination using the gold standard normative score dataset 166. In some embodiments, more than one machine learning model may be trained for normative score determination, either on the same dataset or on a different dataset. For example, different models may be trained to score different assessment activities.

In some embodiments, the method 300 further collecting gold standard cognitive normative scores for the dataset 166. The gold standard cognitive scores may comprise normative scores assigned by trained experts to patients performing at least one cognitive assessment activity. In some embodiments, a first dataset of normative scores is collected alongside the gold standard speech features and cognitive measurements, that is, the trained expert assigns a score to a given cognitive assessment activity as the activity is performed. A second dataset may be collected from care provider users using the system 100 who enter their own scores via the user interface application 111. The first and second datasets may be used to train and monitor model performance and drift as discussed above for the gait/balance normative scoring model 153.

At block 308, at least one outcome prediction model 157 is trained for patient outcome prediction using the gold standard patient outcomes dataset 168. In some embodiments, more than one machine learning model may be trained for outcome prediction, either on the same dataset or on a different dataset. For example, different models may be trained to predict different types of patient outcomes.

In some embodiments, the method 300 further comprises obtaining the gold standard patient outcome dataset 168 by collecting data provided by patient and/or care provider users of the system 100, via the user interface application 111, regarding patient outcomes in association with previous cognitive and/or gait/balance assessments for that patient including measurements and/or normative scores. The data may include measurements and normative scores for individual assessments as well as dual-tasking assessments, including cost factors. Thus, the outcome prediction models 157 may be trained to generate predictions based on both gait/balance and cognitive assessment data from both individual and dual-task assessments.

In some embodiments, cognitive and gait/balance measurements and normative scores, as well as patient outcomes, may be collected from diverse populations including healthy individuals as well as individuals with a variety of conditions that affect gait/balance and/or cognition. Such a large breadth of data may improve the accuracy and sensitivity of the patient outcome predictions.

As discussed above, the outcome prediction models 157 may initially be trained using both extracted measurements and normative scores, as the dataset grows, the models 157 may be trained to generate predictions directly from one or more extracted measurements.

The outcome prediction model(s) 157 may be trained using a train-validation-test split of the dataset 158 or by any other suitable machine learning or deep learning training methodology. The trained model(s) 157 may then be monitored for model performance over time to ensure the model remains relevant and accurate.

Although the steps of the method 300 are shown in a particular order in FIG. 7, it will be understood that the models in each step may be trained simultaneously or in different orders.

Therefore, embodiments of the systems and methods disclosed herein allow highly sensitive, accurate, objective, precise and reliable gait, balance, and cognitive assessments to be performed using user devices, such as smart phones, tablets, and cameras connected to laptops/desktops, rather than relying on specialized and expensive equipment such as motion capture systems or more subjective observations made by clinicians. The disclosed systems and methods improve accessibility for patients and providers, and allow assessments to be conducted in typical clinical settings or at home. The assessments can also be done by a care provider, including an untrained care provider such as a family member, or the by patient themselves. Patient self-assessments are particularly advantageous for patients in remote locations or who have difficulty traveling. Further, the systems and methods allow for logging of audit trails compliant with regulatory bodies.

The disclosed systems and methods may also provide predictions for complex and multi-factorial patient outcomes such as long-term improvements or deterioration in gait or balance as well as diagnoses, prognosis and recovery from specific health conditions without performing a large battery of different assessments, under the observation of a trained clinician, as would be performed today. For example, a single dual-task assessment with one gait/balance assessment activity and one cognitive assessment activity may allow for prediction of a complex patient outcome such as fall risk. In addition, direct measurements of gait such as various step and stride measurements may be used to predict outcomes more accurately than complex assessment activities. The inclusion of cognitive factors improves the sensitivity and accuracy of outcome predictions related to aging, or for various health conditions, such as neurodegenerative disorders or concussions, that have both cognitive and gait/balance components. It also increases the utility of the assessment system, as gait/balance and cognition are typically assessed together, as part of clinical neuromotor assessments, but current measurement systems focus on either one or the other. In addition, inclusion of dual-task assessments may improve sensitivity and facilitate detecting and tracking subtle changes earlier and over time.

Embodiments of the systems and methods herein are also able to process complex video and audio data more efficiently and generate patient outcome predictions more accurately and precisely than conventional methods. A single video/audio recording may be processed to provide pose information and speech information that may be used for multiple different measurements and/or normative scores, which are then used as input for the patient outcome models. Use of the audio data to pre-process the video data also helps to improve the efficiency of subsequent models.

Although particular embodiments have been shown and described, it will be appreciated by those skilled in the art that various changes and modifications might be made without departing from the scope of the disclosure. The terms and expressions used in the preceding specification have been used herein as terms of description and not of limitation, and there is no intention in the use of such terms and expressions of excluding equivalents of the features shown and described or portions thereof. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A computer-implemented method comprising:
receiving, from a user device, video data and audio data of a patient assessment, wherein the patient assessment comprises at least one gait or balance assessment activity and/or at least one cognitive assessment activity performed by a patient;
processing the video data to obtain pose information, wherein the pose information comprises a plurality of joint points representing the patient;
processing the audio data to obtain speech information in the form of transcribed text, wherein the speech information comprises semantic information and/or speech features;
extracting at least one gait or balance measurement from the pose information;
extracting at least one cognitive measurement or cognitive normative score from the speech information; and
generating, via a machine learning model, a prediction of a patient outcome using the at least one gait or balance measurement and/or the at least one cognitive measurement or cognitive normative score as input, wherein the machine learning model is trained using training data comprising patient outcomes in association with a plurality of previous gait or balance assessments and cognitive assessments.

2. The method of claim 1, further comprising determining a gait or balance normative score for the at least one gait or balance assessment activity based on the at least one gait or balance measurement; and wherein the input for the machine learning model further comprises the gait or balance normative score.

3. The method of claim 1, wherein the cognitive normative score is extracted from the semantic information of the speech information.

4. The method of claim 3, further comprising determining at least one additional cognitive normative score based on the at least one cognitive measurement; and wherein the input for the machine learning model further comprises the at least one additional cognitive normative score.

5. The method of claim 1, wherein the input for the machine learning model further comprises at least one of the pose information and the speech information.

6. The method of claim 1, wherein the patient outcomes in the training data comprise patient-reported outcomes.

7. The method of claim 1, wherein the at least one gait or balance measurement comprises at least one of: step and stride measurements; gait cycle phases for each leg; estimation of trunk sway; asymmetry detection during movement; joint angles; and joint velocities.

8. The method of claim 1, wherein at least one of processing the video data and extracting the at least one gait or balance measurement are performed using at least one machine learning model trained using sensor data collected from physical sensors during a plurality of gait or balance assessments.

9. The method of claim 1, wherein at least one of processing the audio data and extracting the at least one cognitive measurement or cognitive normative score are performed using at least one machine learning model trained using transcribed and labeled text collected from a plurality of cognitive assessments conducted by trained experts.

10. The method of claim 9, wherein the at least one machine learning model is a large-language model.

11. The method of claim 1, wherein processing the audio data further comprises identifying one or more speech cues in the speech information and assigning a respective timestamp to each of the one or more speech cues.

12. The method of claim 11, wherein processing the video data further comprises cropping or segmenting the video data based on the respective timestamp of at least one speech cue of the one or more speech cues.

13. The method of any one of claim 11, wherein the at least one gait or balance measurement comprises a timed measurement and one speech cue of the one or more speech cues indicates the start of the at least one gait or balance assessment activity, and wherein processing the video data further comprises assigning an activity start timepoint to the video data based on the respective timestamp of the one speech cue.

14. The method of claim 13, wherein the timed measurement comprises a measured time lag between the activity start timepoint and a movement start timepoint, wherein the movement start timepoint is assigned based on the pose information.

15. The method of claim 1, wherein the at least one gait or balance assessment activity and the at least one cognitive assessment activity are performed simultaneously as a dual-task assessment.

16. The method of claim 15, further comprising determining a respective cost factor for each of the at least one gait or balance assessment activity and the at least one cognitive assessment activity in comparison to another gait or balance assessment activity and cognitive assessment activity, respectively, performed independently.

17. A computer-implemented method comprising:
receiving, from a user device, video data and audio data of a patient assessment, wherein the patient assessment comprises at least one gait or balance assessment activity;
processing the audio data to obtain speech information in the form of transcribed text, wherein processing the audio data further comprises identifying one or more speech cues in the speech information and assigning a respective timestamp to each of the one or more speech cues;
assigning one or more timepoints to the video data based on the respective timestamps of the one or more speech cues to define at least one segment;
processing the video data of the at least one segment to obtain pose information, wherein the pose information comprises a plurality of joint points representing the patient;
extracting at least one gait or balance measurement from the pose information; and
generating, via a machine learning model, a prediction of a patient outcome using the at least one gait or balance measurement as input, wherein the machine learning model has been trained using training data comprising patient outcomes in association with a plurality of previous gait or balance assessments.

18. The method of claim 17, further comprising extracting at least one cognitive measurement or cognitive normative score from the speech information, and wherein the input for the machine learning model further comprises the at least one cognitive measurement or cognitive normative score.

19. The method of claim 17, wherein:
the at least one gait or balance assessment activity is performed simultaneously with at least one cognitive assessment activity;
the method further comprises determining a cost factor for the at least one gait or balance assessment activity in comparison to another gait or balance assessment activity performed independently; and
the input for the machine learning model further comprises the cost factor.

20. A system comprising:
an electronic medical records (EMR) subsystem comprising one or more databases to receive and store video and audio data of patient assessments and patient assessment results; and
a data analysis and prediction subsystem comprising one or more processors executing processor-readable instructions causing the one or more processors to perform the method of claim 1.
